(19) United States
(12) Patent Application Publication
(10) Pub. No.: US 2026/0232542 A1
(43) Pub. Date: Aug. 13, 2026

(54) DENTAL COMPOSITE MATERIAL CONTAINING PHOTOINITIATORS

(71) Applicant: Kulzer GmbH, Hanau (DE)

(72) Inventors: Christoph MEIER, Bruchköbel (DE); Andreas UTTERODT, Neu-Anspach (DE); Raif KOCOGLU, Wölfersheim (DE)

(73) Assignee: Kulzer GmbH, Hanau (DE)

(21) Appl. No.: 19/156,584

(22) PCT Filed: Feb. 13, 2024
(86) PCT No.: PCT/EP2024/053547

§ 371 Date: Aug. 14, 2025

(30) Foreign Application Priority Data
Feb. 16, 2023 (DE) 10 2023 103 892.3

(51) Int. Cl. A61K 6/893; A61K 6/30; A61K 6/62; A61K 6/77

(52) U.S. Cl. CPC A61K 6/893; A61K 6/30; A61K 6/62; A61K 6/77

(57) ABSTRACT

Polymerizable, light-radiation curable dental composite materials are provided and comprise (i) 30 to 90 wt. % of at least one inorganic filler component comprising at least one dental glass and optionally at least one amorphous metal oxide, (ii) 5 to 60 wt. % of at least one urethane acrylate, urethanemethacrylate or mixtures thereof, (iii) 0.01 to 30 wt. % of at least one di-, tri-, tetra- or multifunctional monomer which is not urethane acrylate and/or urethane alkylacrylate, and (iv) 0.01 to 10 wt. % comprising hexaarylbisimidazol (HABI) and/or substituted hexaarylbisimidazol and comprising at least one mercaptotetrazol as the initiator system, and optionally at least one pigment, wherein the total composition of the composite material is 100 wt. %.

### SUMMARY

The invention relates to a polymerizable, light-curable dental composite material comprising
(i) 30 to 90% by weight of an inorganic filler component comprising at least one dental glass and, optionally, at least one amorphous metal oxide,
(ii) 5 to 60% by weight comprising at least one urethane acrylate, urethane methacrylate or mixtures thereof,
(iii) 0.01 to 30% by weight of at least one di-, tri-, tetra- or multi-functional monomer which is not a urethane acrylate and/or a urethane alkyl acrylate,
(iv) 0.01 to 10% by weight comprising hexaarylbisimidazole (HABI) and/or substituted hexaarylbisimidazole and comprising at least one mercaptotetrazole as initiator system and optionally at least one pigment, the total composition of the composite material being 100% by weight (wt.-%), and a polymerized composite material for the production of direct dental prostheses or indirect dental prostheses.

The invention relates to a polymerizable dental composite material that can be cured with light rays, comprising
(i) 30 to 90 wt.-% (% by weight) of an inorganic filler component comprising at least one dental glass and, optionally, at least one amorphous metal oxide,
(ii) 5 to 60% by weight comprising at least one urethane acrylate, urethane methacrylate or mixtures thereof,
(iii) 0.01 to 30 wt.-% of at least one di-, tri-, tetra- or multi-functional monomer that is not a urethane acrylate and/or urethane alkyl acrylate, in particular urethane methyl acrylate,
(iv) 0.01 to 10% by weight comprising hexaarylbisimidazole (HABI) and/or substituted hexaarylbisimidazole and comprising at least one mercaptotetrazole as an initiator system, and optionally
(v) 0.01 to 10 wt.-% of at least one stabilizer and optionally at least one UV additive,
(vi) optionally at least one pigment, wherein the total composition of the composite material is 100 wt.-%, as well as a polymerized composite material for the production of direct dental prostheses or indirect dental prostheses, as well as composite material for use in the production of direct dental prostheses or indirect dental prostheses. The composite material according to the invention has a high curing depth and, at the same time, high flexural strength and a high modulus of elasticity.

Many dental composites are known that are universally applicable for direct adhesive restoration as well as for the extraoral manufacture of indirect dental prostheses. US2017/0266081A1 and US2021/0161772A1 disclose a photopolymerizable dental material containing an ethylenically unsaturated functional group and a hexaarylbisimidazole derivative. These documents disclose, in the examples, MMT (3-mercapto-4-methyl-4H-1,2,4-triazole), MBT (2-mercaptobenzothiazole) and DMABE (dimethylamino-benzoic acid ethyl ester), which are used as co-initiators for different o-CI-HABI derivatives. Although good mechanical properties are obtained according to the examples, the cure depths are low (≤3 mm, see Table 2).

Modern dental lamps emit blue light with emission maxima in the wavelength range from 400 nm to 500 nm. A typical photoinitiator (PI) that absorbs optimally in this wavelength range and is capable of forming radicals is camphorquinone (CQ). However, since the photochemically formed radicals of CQ recombine quickly, CQ shows only very low activity of pure CQ in combination with acrylates, methacrylates, vinylenes, or other radically polymerizable double bonds. An established method for better utilizing the reactivity of CQ radicals in the aforementioned wavelength range is the addition of a tertiary amine, which forms a CQ radical and a radical of the tertiary amine via intermolecular hydrogen radical transfer according to Norris Type II. Intramolecular recombination of CQ is excluded and the radical polymerization of polymerizable double bonds can be initiated with the help of the radicals formed.

Aminobenzoates such as 2-ethylhexyl-4-(dimethylamino)benzoate, 2-butoxyethyl-4-(dimethylamino)benzoate or others are preferred as co-initiators. Experience has shown that these co-initiators can be used to achieve high double bond conversion rates, high mechanical strength (flexural strength, modulus of elasticity, fracture toughness, etc.) and high cure depths in dental restorative composites. This was also achieved in the aforementioned US2017/0266081A1 and US2021/0161772A1.

However, 2-ethylhexyl-4-(dimethylamino)benzoate was recently classified by the European Chemical Agency (ECHA) as potentially harmful to fertility and thus declared a Class 1B CMR substance, which calls into question the general use of this class of substances in medical devices.

The use of hexaarylbisimidazoles as photoinitiators and their combination with thiols or thiol heterocycles is generally known in the literature (J. Lalevée, J.-P-Fouassier; Dyes and Chromophores in Polymer Science, Wiley; 2015; Chapter 4.1; p. 130-131, R. Dessauer; Photochemistry, History and Commercial Applications of Hexaarylbiimidazoles: All about HABIs, Elsevier, 2006, R. Dessauer; The invention of Dylux instant-access imaging materials and the development of HABI chemistry—a personal history; Adv. Photochem., 2005, 28, 129-261 and B. M. Monroe and G. C. Weed, Photoinitiators for free radical-initiated photoimaging systems, Chem. Rev., 1993, 93, 435-448). Berdzinski et al. were able to demonstrate, in particular using photo-DSC, that mercaptotriazole or phenyl-mercapto-triazole in combination with chlorine-substituted hexaarylbisimidazoles (o-CI-HABI) exhibit high reactivity compared to 1,6-hexanediol diacrylate (S. Berdzinski, N. Strehmel, H. Lindauer, V. Strehmel, B. Strehmel; Photochem. Photobiol. Sci., 2014, 13, 789).

The objective of the invention was to provide an alternative photoinitiator system that offers equivalent or better properties than the known camphorquinone (CQ) aminobenzoate photoinitiator system in terms of the curing depth of the dental material and/or the mechanical properties of the dental material that can be obtained. Therefore, a further general task was to provide an alternative photoinitiator system that exhibits good, equivalent or better properties than a CQ/aminobenzoate PI system in conventional dental restorative composites. A further task was to provide a photoinitiator system that is free of aminobenzoates. In particular, the initiator system should exhibit good compatibility with conventional dental compositions, which, depending on the dental material, may have low to very high filler contents, in particular comprising dental glasses and metal oxides, etc., depending on the specific dental application. Furthermore, the task was to provide a photoinitiator system that is compatible with the monomers or monomer mixtures used. Another task was to keep the content of the initiator system as low as possible.

The invention relates to a polymerizable, light-curable dental composite material according to one of claims 1 to 14, a polymerized composite material according to one of claim 15 or 16, and the use according to claim 17, as well as a photoinitiator system according to claim 18. The invention is described in the claims and in detail in the description and the embodiments.

Surprisingly, it was found that high cure depths can be achieved with different aromatic mercaptotetrazoles in combination with HABI, in particular with o-CI-HABI. In contrast, the cure depths of the mercaptotriazole compound, as described above, are less than or equal to 3.00 mm in the prior art.

A preferred subject matter of the invention comprises a photoinitiator system for use in polymerizable, light-curable compositions, in particular comprising at least one compound comprising at least one ethylenic group, preferably dental composite material comprising
a) hexaarylbisimidazole and/or substituted hexaarylbisimidazole and
b) at least one mercaptotetrazole comprising phenyl and/or substituted phenyl substituted mercaptotetrazole as an initiator system, and optionally
c) at least one mercaptotriazole and/or substituted mercaptotriazole, wherein c) and b) are preferably present in a weight ratio of 5:3 to 3:5, in particular in a weight ratio of 1:3 to 3:5, c) and b) are particularly preferred in a weight ratio of 1:3, a:(b+c) being particularly preferred in a weight ratio of greater than or equal to 1.1:1.

A combination of HABI and mercaptotetrazole(s) achieves a significantly improved cure depth in the dental composite material. With the combination of HABI, mercaptotetrazole(s) and mercaptotriazole, a significantly improved curing depth can be achieved with further improved mechanical properties, such as improved flexural strength and an improved modulus of elasticity.

The invention relates to a polymerizable, light-curable dental composite material comprising
(i) 30 to 90 wt.-% of an inorganic filler component comprising at least one dental glass and, optionally, at least one amorphous metal oxide,
(ii) 5 to 60 wt.-% comprising at least one urethane acrylate, urethane alkyl acrylate, in particular urethane methacrylate, or mixtures thereof,
(iii) 0.01 to 30 wt.-% of at least one di-, tri-, tetra- or multi-functional monomer which is not urethane acrylate and/or urethane alkyl acrylate, in particular urethane methacrylate,
(iv) 0.01 to 10 wt.-% comprising hexaarylbisimidazole (HABI) and/or substituted hexaarylbisimidazole and comprising at least one mercaptotetrazole as an initiator system, wherein the total composition of the composite material is 100 wt.-%. Furthermore, the dental composite material may comprise mixtures of hexaarylbisimidazoles (HABI) and/or substituted hexaarylbisimidazoles and mixtures of mercaptotetrazoles as the initiator system.

A preferred combination is iv) comprising a) hexaarylbisimidazole (HABI) and/or substituted hexaarylbisimidazole (substituted HABI), in particular halogen-substituted hexaarylbisimidazole, particularly preferred chlorine-substituted hexaarylbisimidazole, and b) at least one mercaptotetrazole comprising phenyl- and/or substituted phenyl-substituted mercaptotetrazole, in particular with p-substituted phenyl groups substituted mercaptotetrazoles, as initiator system. The hexaarylbisimidazole (HABI) corresponds to 2,2′-bis(phenyl)-4,4′,5,5′-tetraphenyl-1,1′-bi-1H-imidazole. The substituted hexaarylbisimidazole corresponds to a substituted 2,2′-bis(phenyl)-4,4′,5,5′-tetraphenyl-1,1′-bi-1H-imidazole, such as in particular a halogen, in particular fluorine, bromine or chlorine-substituted hexaarylbisimidazole, wherein it is preferred that at least one or two phenyl groups are substituted with 2-chloro, 2,4-dichloro.

Hexaarylbisimidazole (HABI) and/or at least one substituted hexaarylbisimidazole may also be present independently as mixtures. Furthermore, at least one mercaptotetrazole comprising phenyl and/or substituted phenyl-substituted mercaptotetrazoles may also be present independently as mixtures.

A monomer that is not a urethane acrylate and/or a urethane alkyl acrylate, in particular a urethane methacrylate, preferably has at least one ethylenic group, particularly preferably at least two ethylenic groups.

Preferably, the weight ratio of a) hexaarylbisimidazole (HABI) and/or substituted hexaarylbisimidazole to b) mercaptotetrazole is 3:1 to 1:1, in particular from 2.5:1 to 1.1:1. Furthermore, it is preferred that the initiator system (iv) is present in the dental composite material in an amount of 0.1 to 1.0 wt.-%, preferably from 0.1 to 0.7 wt.-%, in the dental composite material.

According to a preferred alternative, the dental composite material comprises (v) 0.01 to 10 wt.-%, in particular 0.01 to 5 wt.-%, preferably from 0.1 to 1 wt.-%, of at least one stabilizer and optionally at least one UV additive, wherein the total composition of the composite material being 100 wt.-%. The UV additives referred to here are organic and inorganic compounds that can be used as UV filters in dental composites. The UV additive oxybenzone can be used as an organic UV filter. This filter mainly absorbs UVB rays and short UVA rays. Further UV additives comprise at least one benzophenone derivative, preferably alkoxy-substituted benzophenone and/or phenol derivative, such as 2-hydroxy-4-methoxybenzophenone, 2,6-bis(1,1-dimethyl)-4-methylphenol, butylphenol (MBBT) and/or tris-biphenyl triazine (TBPT) as well as mixtures comprising at least two of the UV additives, wherein further UV additives known to the skilled person comprising organic and inorganic compounds which act as UV filters can be used.

According to a further embodiment, the dental composite material in (iv) comprises
a) hexaarylbisimidazole and/or substituted hexaarylbisimidazole, in particular chlorine-substituted hexaarylbisimidazole, and
b) at least one mercaptotetrazole, in particular comprising at least one mercaptotetrazole substituted with phenyl and/or at least one mercaptotetrazole substituted with substituted phenyl, and
c) at least one mercaptotriazole and/or at least one substituted mercaptotriazole as the initiator system. Preferred c) mercaptotriazoles and/or substituted mercaptotriazoles comprise at least one mercaptotriazole comprising 3-mercapto-1,2,4-triazoles (MTA), 3-mercapto-4-methyl-4H-1,2,4-triazole and/or mixtures thereof.

The invention also relates to a dental composite material comprising (iv) a) hexaarylbisimidazole (2,2′-bis(phenyl)-4,4′,5,5′-tetraphenyl-1,1′-bi-1H-imidazole) and/or substituted hexaarylbisimidazole (substituted 2,2′-bis(phenyl)-4,4′,5,5′-tetraphenyl-1,1′-bi-1H-imidazole) and b) at least one mercaptotetrazole, in particular selected from 1-phenyl mercaptotetrazoles and p-substituted 1-phenyl mercaptotetrazoles or mixtures of these mercaptotetrazoles, in particular mixtures of the aforementioned mercaptotetrazoles, and c) at least one mercaptotriazole as initiator system.

Preferred mercaptotriazoles, in particular 1-phenyl-mercaptotetrazoles and p-substituted 1-phenyl-mercaptotetrazoles, comprising 5-Mercapto-1-phenyl-1H-tetrazole (MPHTA), 1-(4-hydroxyphenyl)-5-mercapto-1H-tetrazole (HPMTA), 1-(4-ethoxyphenyl)-5-mercapto-1H-tetrazole (EPMATA), 1-(4-carboxyphenyl)-5-mercapto-1H-tetrazole, 4-(5-sulfanyl-1H-1,2,3,4-tetrazol-1yl)benzonitrile (STABN), 1-[4-(5-Mercapto-1H-tetrazol-1-yl)phenyl]ethanone (MTPE) and/or mixtures comprising at least two of the aforementioned tetrazoles.

The invention also relates to a dental composite material comprising as a) substituted hexaarylbisimidazole, which may comprise at least one 2,2′-bis(2-chlorophenyl)-4,4′,5,5′-tetraphenyl-1,1′-bi-1H-imidazole (o-CI-HABI), 2,2′-bis(2,4-dichlorophenyl)-4,4′,5,5′-tetraphenyl-1,1′-bi-1H-imidazole (2,4-CI-HABI), 2,2′-Bis(3-chlorophenyl)-4,4′,5,5′-tetraphenyl-1,1′-bi-1H-imidazole (3-CI-HABI), 2,2′-Bis(4-chlorophenyl)-4,4′,5,5′-tetraphenyl-1,1′-bi-1H-imidazole (4-CI-HABI), 2,2′-Bis(phenyl)-4,4′-bi (2-chlorophenyl)-5,5′-biphenyl-1,1′-bi-1H-imidazole, 2,2′-Bis(phenyl)-4,4′-biphenyl-5,5′-bi (2-chlorophenyl)-1,1′-bi-1H-imidazole, 2,2′-Bis(phenyl)-4,4′,5,5′-tetra(2-chlorophenyl)-1,1′-bi-1H-imidazole and mixtures comprising at least two of the aforementioned hexaarylbisimidazoles. Despite the designation “bis”, the hexaarylbisimidazoles may comprise both homodimers and heterodimers. The compounds may be present as tautomers, conformers, and structural isomers.

The content of hexaarylbisimidazole and/or substituted hexaarylbisimidazole in the dental composite material may be 0.10 to 5 wt.-%, in particular 0.15 to 5 wt.-%, as well as all contents therebetween. The particularly preferred content of at least one hexaarylbisimidazole and/or substituted hexaarylbisimidazole in the dental composite material can be from 0.12 to 0.5, preferably from 0.15 to 0.5 wt.-%, particularly preferably from 0.015 to 0.25 wt.-%.

A particularly preferred dental composite material comprises as b) the at least one mercaptotetrazole at least one 5-mercapto-1-phenyl-1H-tetrazole (MPHTA), 1-(4-hydroxyphenyl)-5-mercapto-1H-tetrazole (HPMTA), 1-(4-ethoxyphenyl)-5-mercapto-1H-tetrazole (EPMATA), 1-(4-carboxyphenyl)-5-mercapto-1H-tetrazole, 4-(5-sulfanyl-1H-1,2,3,4-tetrazol-1yl)benzonitrile (STABN), 1-[4-(5-Mercapto-1H-tetrazol-1-yl)phenyl]ethanone (MTPE) and/or mixtures comprising at least two of the aforementioned tetrazoles, preferably comprising b) 5-Mercapto-1-phenyl-1H-tetrazole (MPHTA), 1-(4-hydroxyphenyl)-5-mercapto-1H-tetrazole (HPMTA), 1-(4-Ethoxyphenyl)-5-mercapto-1H-tetrazole (EPMATA), 1-(4-carboxyphenyl)-5-mercapto-1H-tetrazole, 4-(5-sulfanyl-1H-1,2,3,4-tetrazol-1yl)benzonitrile (STABN), and/or mixtures of at least two of the aforementioned tetrazoles. Alternatively, the at least one mercaptotetrazole is selected from the aforementioned. Particularly preferred mercaptotetrazoles may comprise 5-Mercapto-1-phenyl-1H-tetrazole (MPHTA), 1-(4-hydroxyphenyl)-5-mercapto-1H-tetrazole (HPMTA), 1-(4-ethoxyphenyl)-5-mercapto-1H-tetrazole (EPMATA) and/or mixtures of at least two of the aforementioned mercaptotetrazoles. The content of at least one mercaptotriazole may be 0.02 to 5 wt.-%, as well as all contents in between. A content of 0.15 to 0.5 wt.-% is preferred.

An alternative subject matter of the invention is a polymerizable, light-curable dental composite material comprising
(i) 40 to 90 wt.-%, in particular 40 to 75 wt.-%, of an inorganic filler component comprising at least one dental glass and optionally at least one amorphous metal oxide,
(ii) 10 to 60 wt.-%, in particular 10 to 30 wt.-%, comprising at least one urethane acrylate, urethane alkyl acrylate, in particular urethane methacrylate, or mixtures thereof,
(iii) 0.01 to 25 wt.-%, in particular 10 to 25 wt.-%, of at least one di-, tri-, tetra- or multi-functional monomer, not being a urethane acrylate and/or not being a urethan alkyl acrylate, in particular urethane methacrylate,
(iv) 0.01 to 10 wt.-% comprising hexaarylbisimidazole (HABI) and/or substituted hexaarylbisimidazole and comprising at least one mercaptotetrazole as an initiator system and optionally at least one pigment, the total composition of the composite material being 100 wt.-%. Furthermore, the dental composite material may comprise mixtures of hexaarylbisimidazoles (HABI) and/or substituted hexaarylbisimidazoles and mixtures of mercaptotetrazoles as the initiator system.

A dental composite material comprising (iv) is particularly preferred
a) 0.1 to 0.5 wt.-%, in particular 0.1 to 0.3 wt.-%, particularly preferred 0.15 to 0.3 wt.-% hexaarylbisimidazole and/or substituted hexaarylbisimidazole and/or mixtures thereof, and
b) 0.02 to 0.2 wt.-%, preferably 0.02 to 0.15 wt.-%, particularly preferably from 0.3 to 0.15 wt.-%, at least one mercaptotetrazole, in particular comprising mercaptotetrazoles substituted with phenyl and/or substituted phenyl and/or mixtures thereof, in particular mercaptotetrazoles substituted with p-substituted phenyl groups, and
c) 0.02 to 0.2 wt.-%, in particular 0.03 to 0.1 wt.-%, at least one mercaptotriazole and/or substituted mercaptotriazole, wherein the total composition of the composite material is 100 wt.-%.

According to a further alternative, a dental composite material is preferred which comprises as (iv) a) 0.15 to 0.3 wt.-% of hexaarylbisimidazole and/or substituted hexaarylbisimidazole and b) 0.04 to 0.2% by weight of at least one mercaptotetrazole, in particular comprising mercaptotetrazoles substituted with phenyl and/or substituted phenyl, in particular mercaptotetrazoles substituted with p-substituted phenyl groups, and
c) 0.03 to 0.1 wt.-% of at least one mercaptotriazole and/or substituted mercaptotriazole, wherein c) and b) are present in a weight ratio of 5:3 to 3:5, in particular in a weight ratio of 1:3 to 3:5, c) and b) are particularly preferred in a weight ratio of 1:3, wherein the total composition of the composite material is 100 wt.-%. All weight ratios, such as 5:3 to 3:5, as well as all other weight ratios, may vary independently by plus/minus 5%, preferably by plus/minus 2.5%, particularly preferably by plus/minus 1%.

If c) and b) are present in a weight ratio of 5:3 to 3:5, in particular in a weight ratio of 1:3 to 3:5, this means the weight ratio of c) to b), as c)/b) in (wt.-%/wt.-%).

Furthermore, the invention relates to a dental composite material comprising (iv)
a) 0.15 to 0.3% by weight of hexaarylbisimidazole and/or substituted hexaarylbisimidazole and
b) 0.06 to 0.2% by weight of at least one mercaptotetrazole, in particular comprising phenyl and/or substituted phenyl-substituted mercaptotetrazoles, in particular from 0.1 to 0.2% by weight, and
c) 0.03 to 0.1 wt.-% of at least one mercaptotriazole and/or substituted mercaptotriazole, wherein c) and b) are present in a weight ratio of 5:3 to 3:5, in particular c) and b) are present in a weight ratio of 1:3 to 3:5, and a:(b+c) are more preferably present in a weight ratio of 1:1 to 2:1, and further preferably in a weight ratio of a:(b+c) greater than or equal to 1.1:1, wherein the total composition of the composite material is 100 wt.-%.

The invention also relates to a dental composite material comprising (iv)
a) 0.15 to 0.3 wt.-% hexaarylbisimidazole and/or substituted hexaarylbisimidazole and b) 0.08 to 0.2% by weight of at least one mercaptotetrazole, in particular comprising phenyl- and/or substituted phenyl-substituted mercaptotetrazoles, in particular from 0.1 to 0.2% by weight, and
c) 0.03 to 0.08 wt.-% of at least one mercaptotriazole and/or substituted mercaptotriazole, wherein c) and b) are present in a weight ratio of 1:3 to 3:5, particularly preferred being a:(b+c) in a weight ratio of greater than or equal to 1.1:1, wherein the total composition of the composite material is 100 wt.-%.

According to a further alternative, the polymerizable composite material may comprise (vi) optionally at least one pigment, dye or a mixture of pigment and dye or also a mixture of pigments and/or dyes. It may be further preferred that the content of (vi) is from 0.001 to 5 wt.-% in the total composition. Dyes that are soluble in the dental composite material, such as those according to DIN 55934, may be particularly preferred.

Furthermore, a polymerized dental composite material is the subject of the invention, in particular a composite material cured or polymerized with light rays, preferably a composite material polymerized with an LED, in particular with emission maxima in the wavelength range from 440 to 550 nm and a light output greater than or equal to 700 mW/cm², preferably greater than or equal to 1000 mW/cm², preferably greater than or equal to 1200 mW/cm².

A particularly preferred polymerized dental composite material has a flexural strength greater than or equal to 130 [MPa] and a modulus of elasticity greater than or equal to 6500 [MPa], determined in accordance with DIN EN ISO 4049:2019. Equally preferred is a modulus of elasticity greater than or equal to 5500 [MPa] at a curing depth greater than or equal to 8.5 mm, preferably a modulus of elasticity greater than or equal to 6000 [MPa] at a curing depth greater than or equal to 9 mm, and particularly preferred is a modulus of elasticity greater than or equal to 6500 [MPa] at a cure depth of greater than or equal to 9.5 mm and optionally a flexural strength of greater than or equal to 135 [MPa], in particular greater than or equal to 140 [MPa] in a polymerized composite material. Polymerization is preferably carried out with a light source such as Translux2Wave, Translux or LED, with emission maxima in the wavelength range from 400 to 550 nm, in particular from 440 to 480 nm, the light output preferably being greater than 700 mW/cm², preferably greater than or equal to 1000 mW/cm², in particular greater than or equal to 1200 mW/cm². The mechanical properties are determined in accordance with the aforementioned standard the polymerization preferably as specified in the examples.

It is also preferred that the curing depth or polymerization depth of the polymerized composite material in mm is greater than or equal to 9.0 mm, wherein the curing depth or polymerization depth is determined in accordance with DIN EN ISO 4049:2019, 7.10, in particular the curing or polymerization takes place for 20 seconds with a light source, in particular an LED light source, such as Translux2Wave, with emission maxima in the wavelength range from 400 to 550 nm, in particular from 440 to 480 nm, and a light output greater than or equal to 1200 mW/cm².A Translux2Wave (light output greater than 1000 to 1200 mW/cm² can be used for curing and determining the double bond conversion. A Translux Wave (light output greater than 700 mW/cm² from Kulzer GmbH can be used for curing and/or determining the mechanical properties; Translux2Wave is preferred.

A high filler content is advantageous in order to achieve very good mechanical properties of the cured composite and at the same time reduce the polymerization shrinkage that occurs during curing. These properties are also decisive for the long-term success of the dental prosthesis material. However, the high filler content generally prevents a high curing depth or polymerization depth during light curing of the composite material.

The conditions listed below were set in the examples below:
The monomers, fillers, and additives used were the same in all tests.
Camphorquinone (CQ) and 2-ethylhexyl 4-(dimethylamino)benzoate (EHA) were used as the reference photoinitiator system.
The new photoinitiator system, synonymous with initiator system, comprises a hexaarylbisimidazole (HABI-1) as photosensitizer.
Furthermore, a combination of co-initiators comprising various mercaptotriazoles and various mercaptotetrazoles in different quantities and mixing ratios is added. The “best” co-initiators for high double bond conversion, high mechanical strength, and high cure depth have been identified, and the best combination of these co-initiators and the optimal mixing ratio of these, in combination with the photosensitizer HABI, in particular o-CI-HABI, has been determined.

In addition, the proportion of the photosensitizer hexaarylbisimidazole (HABI), in particular substituted hexaarylbisimidazole, was evaluated in terms of high mechanical strength and high cure depth.

Depending on the desired filler content, the particle size distribution can be rather broad for high packing density and excellent mechanical properties, or rather narrow for specific applications. The mean value of the particle size distribution can be in the range of 0.1 μm to 30 μm, preferably in a range of 0.5 μm to 20 μm. The particle size distribution, based on a content of 5 to 75 wt.-% of a dental glass with a particle size distribution of d (50) of a dental glass fraction in the range of 0.7 to 2.0 μm, in particular 1.2 to 2.0 μm (micrometers) relative to the total composition, can be adjusted, preferably with d₅₀ of 1.8 μm with plus/minus 0.25 μm, particularly preferred d₉₉ less than or equal to 20 μm or d₉₉ less than or equal to 10 μm. Alternatively, further dental glass fractions with different smaller and/or larger particle size distributions can be added in order to optimally adjust the packing density. The coordinated packing density enables optimal adjustment of the mechanical properties and reduced shrinkage. The particle size distribution can be determined by laser diffraction.

Particle sizes are usually determined by laser diffraction, for example with a Mastersizer 3000E, by measuring the intensity of the scattered light from a laser beam as it passes through a dispersed particle sample. The analysis can be performed using Mie and Fraunhofer scattering.

In the analysis, the size of the particles is calculated from the diffraction pattern generated using the data obtained. This is done by analyzing the data of the angle-dependent scattered light intensity and using it as the basis for calculating the size of the particles responsible for the diffraction pattern according to Mie theory. The particle size is given as the diameter of the sphere with the same volume. Thus, the particle size distributions revealed can be understood as the particle size distribution of the sphere with the same volume.

A urethane acrylate preferably comprises a difunctional urethane acrylate, which is preferably selected from difunctional urethane acrylates with a bivalent alkylene group, preferably comprising difunctional urethane alkyl acrylates with a bivalent alkylene group with alkyl 1 to 10 C atoms and alkylene 3 to 20 C atoms.

A photochemically polymerizable dental composite is understood to be a composite material that is polymerizable by means of UV emission and/or visible light (VIS emission), preferably a composite material polymerizable by means of a radiation source with emission maxima in the spectral range from 400 nm to 530 nm, preferably with at least one maximum or maxima in the spectral range from 440 to 500 nm. It is particularly preferred that the composite material is irradiated for 10 seconds or longer, especially per projection surface of the radiation source. Irradiation for 15 seconds to 5 minutes, preferably for 10 to 30 seconds per projection surface of the radiation source, is further preferred. All conventional radiation sources with an emission wavelength, preferably emission maxima in the spectral range from 440 to 480 nm and an intensity greater than 500 mW/cm², in particular greater than or equal to 1200 mW/cm², are suitable as radiation sources, in particular as used in the dental field. A radiation source with an LED light source is particularly preferred.

The invention also relates to a dental composite material obtainable by polymerization with a UV and/or Vis radiation source, preferably with a Vis radiation source with emission maxima in the spectral range from 380 nm to 530 nm, preferably with at least one maximum or maxima in the spectral range from 400 to 500 nm.

In one embodiment, it is particularly preferred if the inorganic filler component consists of at least one dental glass or a mixture of dental glasses, in particular of the aforementioned medium particle size, and an amorphous metal oxide, in particular a non-agglomerated amorphous metal oxide, preferably a silanized amorphous metal oxide. The dental glass may preferably also be silanized. The silanization preferably comprises acrylic functionalization. The amorphous metal oxide may comprise silicon dioxide, precipitated silicon dioxide, pyrogenic silica, zirconium oxide, mixed oxides or mixtures thereof, in particular the metal oxides are silanized.

The following are preferred dental glasses: Aluminum silicate glasses or fluoroaluminum silicate glasses, fluoroaluminum silicate glasses containing boron, barium aluminum silicate, strontium silicate, strontium borosilicate, lithium silicate, and/or lithium aluminum silicate, as well as mixtures of at least two of the aforementioned dental glasses. Amorphous spherical fillers based on oxide or mixed oxides, such as amorphous SiO₂, ZrO₂ or mixed oxides of SiO₂ and ZrO₂ can be used as amorphous metal oxides or as mixtures of amorphous metal oxides.

The invention also relates to a dental composite material comprising
a) a dental glass with a mean particle size d₅₀ of 1.8 μm (micrometer) with plus/minus 0.25 μm and preferably d₉₉ less than or equal to 20 μm, in particular d₉₉ less than or equal to 10 μm, or b) a dental glass comprising a mixture of dental glasses of different fractions with average particle sizes with i) d₅₀ from 2 to 8 μm optionally with plus/minus 0.5 μm, in particular with 4 to 6 μm optionally with plus/minus 0.25 μm, ii) d₅₀ from 1.0 to 2.0 μm optionally with plus/minus 0.25 μm, in particular with 1.2 to 2.0 μm optionally with plus/minus 0.5 μm, preferably with 1.5 μm optionally with plus/minus 0.15 μm, and iii) d₅₀ from 0.5 μm to 1.2 μm optionally with plus/minus 0.15 μm, 0.7 to 0.9 μm optionally with plus/minus 0.5 μm, wherein the fractions of i) to ii) to iii) are present in a ratio of 1 to 4:1:4 to 8, in particular of 2 to 3:1:6 to 7. Particularly preferred are i) d₅₀ of 5 μm optionally with plus/minus 0.5 μm, ii) d₅₀ of 1.8 μm optionally with plus/minus 0.25 μm and iii) d₅₀ of 0.85 μm optionally plus/minus 0.15 μm, wherein the fractions of i) to ii) to iii) are present in a ratio of 1 to 4:1:4 to 8, in particular of 2 to 3:1:6 to 7.

According to a preferred embodiment, the dental composite material comprises at least one dental glass, in particular an X-ray-opaque dental glass, having a mean particle size d₅₀ of 1.2 to 2.0 μm, preferably with a mean particle size of 1.35 to 1.95 μm, in particular with d₅₀ of 1.8 μm, optionally plus/minus 0.15 μm, and preferably with d₉₉ less than or equal to 10 μm. It is particularly preferred that a dental glass with a mean particle size of d₅₀ of approximately 0.85 μm, optionally plus/minus 0.1 μm, in particular plus/minus 0.05 μm, preferably plus/minus 0.03 μm, and preferably with dog less than or equal to 10 μm, is additionally present. A particularly preferred dental glass comprises barium aluminum borosilicate glass. Furthermore, a barium aluminum silicate glass with a refractive index of n=1.52 to 1.55, preferably 1.53, is particularly preferred. A particularly preferred particle size distribution can be in the range of d₁₀ greater than or equal to 0.2 μm to d₉₉ less than or equal to 20 μm, preferably less than or equal to 7.5 μm, preferably with d₁₀ greater than or equal to 0.4 μm to d₉₉ less than 7.5 μm and a mean diameter d₅₀ of 0.7 to 7.5 μm.

According to a preferred embodiment, the dental composite material comprises (i) 70 to 85 wt.-% of at least one inorganic filler component, wherein at least one dental glass has a mean particle size d₅₀ of 0.7 to 2.0 μm of greater than or equal to 50 to 80 wt.-% with respect to the composite material with a total composition of 100 wt.-%, in particular from greater than or equal to 55 to 76 wt.-%, preferably greater than or equal to 60 to 75 wt.-%, particularly preferably greater than or equal to 60 to 71 wt.-% in the total composition of 100 wt.-%. Further preferred in combination with an amorphous silicon dioxide with 4 to 7.5 wt.-% in the total composition.

The invention also relates to a dental composite material comprising (i) 70 to 85 wt.-% of at least one inorganic filler component comprising at least one dental glass comprising barium aluminum borosilicate glass, barium aluminum borofluorosilicate glass, in particular silanized, preferably functionalized with methacryloxy-propyl groups, and optionally at least one non-agglomerated amorphous metal oxide with a primary particle size of 2 to 150 nm, in particular 2 to 100 nm, preferably 2 to 45 nm, wherein the amorphous metal oxide comprises silicon dioxide, precipitated silicon dioxide, pyrogenic silica, zirconium oxide, mixed oxides or mixtures thereof, in particular the metal oxides are silanized.

Preferably, the dental composite material comprises as inorganic filler component (i.1) 66 to 84 wt.-% of at least one dental glass, in particular from 68 to 78 wt.-%, alternatively from 75 to 78 wt.-%, and optionally (i.2) 2 to 10 wt.-% of amorphous metal oxide, in particular from 3 to less than 10 wt.-%, preferably 4 to 8 wt.-%, in the total composition (based on 100 wt.-%). The ratio of dental glass to amorphous metal oxide is preferably from 20:1 to 7:1, preferably from 15:1 to 10:1. The total composition comprising (i), (ii), (iii) and (iv) is 100 wt.-% or optionally comprising (i), (ii), (iii), (iv) and optionally (v) and optionally (vi) is 100 wt.-%.

In a preferred alternative, the composite material may comprise a polymeric particulate filler in addition to the inorganic filler component. The total content of such a polymeric, particulate filler may be 0.01 to 15 wt.-%, preferably 0.5 to 10 wt.-%, in the total composition of the composite material of 100 wt.-%. The particle sizes of the polymeric filler are preferably in the range of 10 to 200 micrometers, in particular 30 to 90 micrometers, and especially preferred 20 to 50 micrometers. The polymeric, particulate filler is preferably not spherical. The polymeric, particulate filler is preferably in the form of fragmented polymer.

The di- to dec-functional urethane acrylates or di- to dec-functional urethane alkyl acrylates are used as monomers and do not comprise any peroxy groups.

According to a particularly preferred embodiment, the dental composite material comprises as (ii)
at least one difunctional urethane acrylate with a bivalent alkylene group and/or urethane alkyl acrylate with a bivalent alkylene group with alkyl of 1 to 10 C atoms, preferably with alkyl equal to methyl, and alkylene with 3 to 20 C atoms, preferably of three different urethane (alkyl) acrylates, as well as optionally at least one at least tetra-functional dendritic urethane (alkyl) acrylate, preferably at least one hexa-functional dendritic urethane (alkyl) acrylate, in particular urethane (meth)acrylate.

According to a particularly preferred embodiment, the dental composite material (ii) comprises a mixture of at least two different urethane (meth)acrylates, preferably of three different urethane (meth)acrylates.

The designation (alkyl) acrylate or (meth)acrylate or urethane (alkyl) acrylate with (alkyl) in brackets or urethane (meth)acrylate with (meth) in brackets means that the designation acrylates or urethane acrylates can comprise with and without alkyl groups or methyl groups. The alkyl groups preferably comprise 1 to 10 C atoms, preferably 1 to 2 C atoms in the urethane alkyl acrylates mentioned. The alkyl groups preferably comprise 1 to 10 C atoms, preferably 1 to 2 C atoms in the (alkyl) acrylates mentioned.

Monomers which are not urethane acrylates or urethane alkyl acrylates are understood to be conventional monomers comprising at least one ethylene group, in particular comprising at least two ethylene groups.

The di-functional urethane acrylate, urethane (alkyl) acrylate, urethane (alkyl) acrylate with a bivalent alkylene group or urethane (meth)acrylate with a bivalent alkylene group is preferably selected from linear or branched urethane dimethacrylates functionalized with a bivalent alkylene group, urethane dimethacrylate-functionalized polyethers with alkylene group(s), such as bis(methacryloxy-2-ethoxycarbonylamino)alkylene, bis(methacryl-oxy-2-ethoxy-carbonylamino)-substituted polyalkylene ethers, preferably 1,6-bis(methacryloxy-2-ethoxycarbonylamino)-2,4,4-trimethylhexane, UDMA with the alternative designation HEMA-TDMI. Preferred is a bis(methacryloxy-2-ethoxycarbonylamino)-alkylene, wherein alkylene comprises linear or branched C3 to C20, preferably C3 to C6, such as particularly preferred alkylene substituted with methyl groups, such as HEMA-TMDI. The bivalent alkylene preferably comprises 2,2,4-trimethylhexamethylene and/or 2,4,4-trimethylhexamethylene.

The at least tetra-functional dendritic urethane methacrylate comprises tetra- to deca-functional dendritic urethane methacrylates.

According to a further preferred embodiment, the dental composite material comprises as components (iii) 0.01 to 15 wt.-% of at least one di-, tri-, tetra- or multifunctional monomer which is not a urethane (alkyl) acrylate and may be selected from di-(meth)acrylic esters, di-(meth)acrylic esters of polyethers, bis-(2′-oxa-3′-oxo-pentyl-4′-en)tetrahydrodi-cyclopentadiene and/or bis-(2′-oxa-3′-oxo-pentyl-4′-methyl-4′-en)tetrahydrodicyclopentadiene and isomers thereof, and tri-, tetra- or multifunctional methacrylic esters of polyethers.

Preferably, the content of components (iii) may be from 0.15 to 25 wt.-%, in particular 0.15 to 20 wt.-%, alternatively particularly preferably 10 to 25 wt.-%, wherein the components (iii) preferably comprise di-methacrylic esters of polyethers, such as preferably dimethacrylate-polyethylene glycol, dimethacrylate-polypropylene glycol. Particularly preferred are dimethacrylate-triethylene glycol (TEGDMA), diethylene glycol dimethacrylate (DEGMA) and dimethacrylate-tetraethylene glycol (TEDMA), bis-GMA and/or ethoxylated bis-GMA.

Water can be added to the dental composite material as a stabilizer to improve its consistency and flow properties for processing. Stabilizers are preferably added to the composite material to prevent premature polymerization and to give the material a certain shelf life. As preferred stabilizers, the composite material in component (iv) comprises at least one stabilizer comprising water or a mixture of the three stabilizers. Preferably, the stabilizers are present in 0.01 to 10 wt.-% of the total composition, particularly preferably in 0.7 to 10 wt.-%, especially in 0.5 to 2 wt.-%. Furthermore, it is preferred that the composite material contains 0.01 to 2 wt.-% water as stabilizer, preferably 0.1 to 1.0 wt.-% water.

For optimum adjustment of the color and natural aesthetics of the polymerized composite material, at least one pigment comprising at least one fluorescent pigment and optionally at least one organic color pigment and/or at least one inorganic color pigment, in particular a non-fluorescent color pigment, is added to the composite material. The at least one fluorescent pigment is preferably an organic fluorescent pigment, in particular a non-polymerizable organic fluorescent pigment, optionally comprising aryl carboxylic acid ester, aryl carboxylic acids, coumarin, rhodamine, naphthalinimide or a derivative of the respective substance. Inorganic fluorescent pigments may comprise CaAl₄O₇:Mn²⁺; (Ba0.98Eu0.02) MgAl₁₀O₁₇, BaMgF₄:Eu₂₊, Y(1.995)Ce(0.005)SiO₅.

As pigments, in particular color pigments, the composite may comprise organic pigments as well as inorganic pigments, in particular comprising diethyl 2,5-dihydroxyterephthalate, N,N′-bis(3,5-xylyl) perylene-3,4:9,10-bis(dicarbimide), copper phthalocyanine, titanate pigment, in particular chromium antimony titanate (rutile structure), spinel black, in particular pigments based on iron oxide black (FeO₄), wherein iron (Fe) is partially substituted by chromium and copper or nickel and chromium or manganese, zinc iron chromium spinel, brown spinel; ((Zn,Fe)(Fe,Cr)₂)O₄) cobalt zinc aluminate blue spinel and/or titanium oxide. The pigments comprising fluorescent and color pigments are preferably present in the total composition in an amount of 0.01 to 10 wt.-%, particularly preferably from 0.01 to 5 wt.-%, preferably from 0.01 to 1 wt.-%.

The pigments must be specifically selected for the dental composite composition in order to achieve a homogeneous color in both the polymerizable composite and the polymerized composite. The production of large material blocks also requires coordination with regard to the selection and concentration of pigments in order to avoid undesirable discoloration due to the dimensions of the polymerized material blocks.

According to a further preferred embodiment, the composite material may comprise: (iv) 0.01 to 2 wt.-% of the initiator system, in particular for the UV and/or VIS range (visible light), and 0.01 to 2 wt.-% stabilizer.

The invention also relates to a polymerized dental composite material which is obtainable by polymerizing the composite material i) with a UV and/or VIS radiation source, preferably with a VIS radiation source with emission maxima in the spectral range from 400 nm to 530 nm, preferably with at least one maximum or maxima in the spectral range from 440 to 500 nm.

Preferred is a polymerized dental composite material, in particular for the production of direct or indirect dental prostheses, comprising
(i) 30 to 90 wt.-%, in particular 40 to 90 wt.-%, of an inorganic filler component comprising at least one dental glass and optionally at least one amorphous metal oxide,
(ii) 5 to 60 wt.-%, in particular 10 to 60 wt.-%, comprising at least one polymer based on at least one urethane acrylate, urethane methacrylate or mixtures thereof, and
(iii) 0.01 to 30 wt.-%, in particular 0.01 to 25 wt.-%, of at least one di-, tri-, tetra- or multi-functional monomer, not being a urethane acrylate and/or not being a urethane alkyl acrylate, in particular urethane meth acrylate, and optionally
(iv) 0.01 to 10 wt.-% comprising hexaarylbisimidazole and/or substituted hexaarylbisimidazole and comprising at least one mercaptotetrazole as initiator system and optionally at least one pigment and optionally at least one stabilizer and optionally at least one UV additive, the total composition of the composite material being 100 wt.-%. It is particularly preferred if the modulus of elasticity is greater than or equal to 6500 [MPa], determined in accordance with DIN EN ISO 4049:2019, and/or a flexural strength greater than or equal to 130 [MPa], in particular with a modulus of elasticity greater than or equal to 6700 [MPa], determined in accordance with DIN EN ISO 4049:2019, and/or a flexural strength greater than or equal to 130 [MPa] and a curing depth or polymerization depth greater than or equal to 9.5 mm determined in accordance with DIN EN ISO 4049:2019. The polymerization can be carried out in accordance with the conditions specified above and below.

According to a further embodiment, the invention relates to the use of the dental composite material for the production of dental filling materials, dental prosthetic restorations in primary mold processes (DIN 8580), additive processes, and/or material removal processes, in particular in a process in which the polymerized composite material is processed by means of machining processes such as milling, cutting, such as laser cutting, polishing, breaking, chipping and/or drilling, in particular not on the patient, i.e. not in a surgical, therapeutic or diagnostic process, preferably in a CAD/CAM process, or as a composite material for the production of direct adhesive dental restorations, in particular not on the patient, i.e. not in a surgical, therapeutic or diagnostic procedure, preferably in a CAD/CAM process, as hoof repair material, as bone cement, as bone cement for cementing artificial joint prostheses, orthodontic appliances and instruments. Particularly preferred the composite material can be used with for the production of medical, veterinary or dental prosthetic restorations comprising crowns, inlays, onlays, superstructures, artificial teeth, dental bridges, dental bars, spacers, abutments or veneers. Furthermore, the polymerized composite material can be used for the manufacture of direct dental prostheses, indirect dental prostheses, prosthetic teeth, dental bridges, and/or milling blanks. Additive manufacturing, also known as rapid technologies or generative manufacturing, refers in particular to all 3D printing processes, such as stereolithography (SLA), digital light processing (DLP), continuous liquid interface production (CLIP), daylight polymer printing (DPP), digital light synthesis (DLS), two-photon lithography (2PP), material jetting, powder bed fusion, such as electron beam melting, selective laser melting (SLM), selective laser sintering (SLM), selective heat sintering (SHS), as well as other additive processes known to those skilled in the art in which composite materials can be constructed. The composite material can also be used in a joining process, such as bonding, or a coating process, in particular not in a surgical, therapeutic, or diagnostic process.

The following urethane (meth)acrylates are also preferred as urethane (meth)acrylates according to the invention: (ii) at least one urethane (meth)acrylate, in particular a urethane dimethacrylate, preferably a bis(methacryloxy-2-ethoxycarbonylamino)alkylene, diurethane acrylate oligomers, alkyl-functional urethane dimethacrylate oligomers, aromatically functionalized urethane dimethacrylate oligomers, aliphatic unsaturated urethane acrylates, bis(methacryloxy-2-ethoxycarbonylamino) substituted polyethers, aromatic urethane diacrylate oligomers, aliphatic urethane diacrylate oligomers, aliphatic urethane diacrylates, hexafunctional aliphatic urethane resins, aliphatic urethane triacrylate, aliphatic urethane acrylate oligomer, unsaturated aliphatic urethane acrylates. Preferred are di-functional and multi-functional urethane (meth)acrylates, such as in particular urethane di(meth)acrylates, particularly preferred is at least one (iii) urethane dimethacrylate selected from linear or branched alkyl-functionalized urethane dimethacrylates, urethane dimethacrylate-functionalized polyethers, in particular bis(methacryloxy-2-ethoxycarbonylamino)-alkylene, bis(methacryloxy-2-ethoxycarbonylamino)-substituted polyethers, preferably 1,6-bis(methacryloxy-2-ethoxycarbonylamino)-2,4,4-trimethylhexanes. Suitable urethane (meth) acrylates are available under the following brand names: Ebecryl 230 (aliphatic urethane diacrylate), Actilane 9290, Craynor 9200 (diurethane acrylate oligomer), Ebecryl 210 (aromatic urethane diacrylate oligomers), Ebecryl 270 (aliphatic urethane diacrylate oligomer), Actilane 165, Actilane 250, Genomer 1122 (monofunctional urethane acrylate), Photomer 6210 (CAS No. 52404-33-8, aliphatic urethane diacrylate), Photomer 6623 (hexafunctional aliphatic urethane resin), Photomer 6891 (aliphatic urethane triacrylate), UDMA, Roskydal LS 2258 (aliphatic urethane-e acrylate oligomer), Roskydal XP 2513 (unsaturated aliphatic urethane acrylate). The urethane (meth)acrylates may preferably be selected from the aforementioned urethane (meth)acrylates or from a mixture of at least two different, preferably at least three different, of the aforementioned urethane (meth)acrylates.

The (iii) at least one di-, tri-, tetra- or multi-functional monomer which is not a urethane (alkyl) acrylate, in particular not a urethane (meth)acrylate, is preferably selected from at least one of the following monomers, in particular a mixture of monomers comprising bis-(2′-oxa-3′-oxo-pentyl-4′-ene)tetrahydrodicyclopentadiene (ester of tricyclo[5.2.1.^{02,6}] decandimethanol and two acrylates or bis-(2′-oxa-3′-oxo-pentyl-4′-methyl-4′-en)tetrahydrodicyclopentadiene (ester of tricyclo[5.2.1.^{02,6})]decandimethanol with two methacrylates) and isomers thereof, 1,4-butanediol dimethacrylate (1,4-BDMA) or pentaerythritol tetraacrylate, bis-GMA monomer (bisphenyl-A-glycidyl methacrylate), triethylene glycol dimethacrylate (TEGDMA) and diethylene glycol dimethacrylate (DEGMA), tetraethylene glycol di(meth)acrylate, decanediol di(meth)acrylate, dodecanediol di(meth)acrylate, hexyldecane dioldi(meth)acrylate, trimethylol propane tri(meth)acrylate, pentaerythritol tetra(meth)acrylate and butanediol di(meth)acrylate, ethylene glycol di(meth)acrylate, polyethylene glycol di(meth)acrylate, ethoxylated/propoxylated bisphenol A di(meth)acrylate, a mixture consisting of at least one of these (meth)acrylates and/or copolymers comprising one or at least two of the aforementioned monomers.

Typical difunctional monomers, especially as (iii), also referred to as crosslinkers or multiple crosslinkers, are tri- or tetraethylene glycol di(meth)acrylate, BDMA, 1,4-butanediol dimethacrylate (1,4-BDMA), bis-GMA monomer (bisphenyl-A-glycidyl methacrylate, an addition product of methacrylic acid and bisphenol-A-diglycidyl ether), diethylene glycol di(meth)acrylate, bisphenol-A-di(meth)acrylate, decanediol di(meth)acrylate, Dodecandiol di(meth)acrylate, Hexyldecandiol di(meth)acrylate, as well as Butandiol di(meth)acrylate, Ethylene glycol Polyethylene di(meth)acrylate, glycol di(meth)acrylates, ethoxylated/propoxylated Bisphenol A di(meth)acrylates. The following difunctional monomers can also be added as diluents (low-viscosity acrylates): Tri- and tetra-functional monomers or multiple crosslinkers comprising trimethylolpropane tri(meth)acrylate, tris(2-hydroxyethyl) isocyanurate triacrylate, pentaerythritol tetraacrylate.

The invention also relates to a composite material which preferably additionally contains at least one or more substances from the groups of fillers, pigments, stabilizers, regulators, antimicrobial additives, UV absorbers, thixotropic agents, catalysts and crosslinking agents. Such additives are used in relatively small quantities, as are pigments, stabilizers, and regulators, e.g., a total of 0.01 to 3.0, particularly 0.01 to 1.0 wt.-% based on the total composition of the composite material. Suitable stabilizers include hydroquinone monomethyl ether or 2,6-di-tert-butyl-4-methylphenol (BHT).

Typical UV additives may also comprise 2,6-di-tert-butyl-4-methylphenol (BHT) or hydroquinone monomethyl ether (MEHQ), 2-hydroxy-4-methoxybenzophenone, HALS (hindered amine light stabilizers), benzotriazole ultraviolet absorbers (UVAs) and hydroxy phenyl triazines (HPT). Particularly suitable stabilizers are, for example, oxybenzone, hydroquinone monomethyl ether and/or 2,6-di-tert-butyl-4-methylphenol (BHT).

The following examples are intended to illustrate the invention without limiting it to these examples.

### EXAMPLES OF EMBODIMENTS

### Methods

### Production of the Pastes of the Composite Material

The monomers, initiators, and additives are weighed and homogenized using a speed mixer (Hauschild—DAC 600.1 FVZ) at 1500 revolutions per minute (^{rpm}) at normal pressure for 5 minutes. The fillers are weighed into the monomer mixture one after the other and the mixture is homogenized again in the speed mixer at 1500 rpm for 5 minutes at normal pressure. The composite is then homogenized again using a three-roll mill. The composite is degassed again in the speed mixer at 700 rpm for 5 minutes under vacuum.

### Determination of Flexural Strength and Modulus of Elasticity

The bending bars are manufactured and the flexural strength and modulus of elasticity (modulus of elasticity) are determined in accordance with DIN EN ISO 4049:2019, 7.11 (test specimens:
25±2 mm×2.0±0.1 mm×(2.0±0.1) mm, light source: Kulzer, Translux Wave, 700 mW/cm², 20 s). Full reference is made to DIN EN ISO 4049:2019. Until testing, the polymerized sample body is stored in water at (37±1° C.) for 24 hours.

### Determination of the Curing Depth

The cure depth/polymerization depth is determined in accordance with DIN EN ISO 4049:2019, 7.10 (test specimens: diameter 4 mm and a minimum length of greater than or equal to 15 mm, light source: Translux 2Wave, 1000 to 1200 mW/cm², 20 s). Full reference is made to DIN EN ISO 4049:2019.

### Determination of the Double Bond Turnover U

The double bond turnover U is determined using ATR-IR. A material sample is applied to a 2 mm deep cylindrical mold. The cylindrical mold filled with composite is placed on an ATR crystal, which is now located on the underside of the material sample. An IR spectrum (wavelength range: 4000 cm⁻¹-700 cm⁻¹ is recorded. The composite is then covered on the upper side with a transparent film, irradiated with a light source (light source: Translux 2Wave, 1200 mW/cm²·20 s) and another IR spectrum is recorded 10 minutes after the end of the photopolymerization. The quotient of the integral of the —C═C— double bond signal (~1645 cm⁻¹) before and after photopolymerization gives the residual double bond content, from which the double bond conversion U in % is determined.

$$U = 100 - ( \frac{total area unpolym .}{total area polym .} \cdot 100 )$$

TABLE 1a
Co-initiators used Mercaptotetrazole

| | | |
|---|---|---|
| 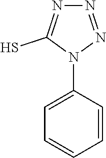 [SMILES: Sc1nnnn1-c1ccccc1] | 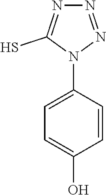 [SMILES: Oc1ccc(-n2nnnc2S)cc1] | 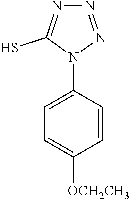 [SMILES: CCOc1ccc(-n2nnnc2S)cc1] |
| 5-Mercapto-1-phenyl-1H- | 1-(4-hydroxyphenyl)-5- | 1-(4-Ethoxyphenyl)-5- |
| tetrazole (MPHTA) | mercapto-1H-tetrazole | mercapto-1H-tetrazole |
| | (HPMTA) | (EPMTA) |
| Co-initiator (tetrazole/+M | Co-initiator (tetrazole/+M | Co-initiator (tetrazole/+M |
| effect/weak) | effect/pronounced) | effect) |

TABLE 1b
Used photosensitizers and co-initiators Mercaptotriazoles

| | | |
|---|---|---|
| 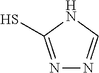 | 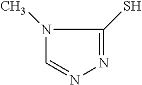 | 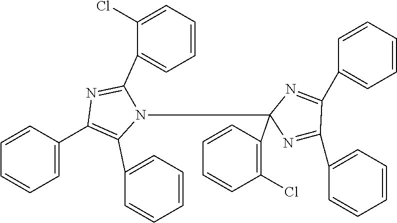 [SMILES: Clc1ccccc1-c1nc(-c2ccccc2)c(-c2ccccc2)n1C1(c2ccccc2Cl)N=C(c2ccccc2)C(c2ccccc2)=N1] |
| 3-Mercapto-1,2,4- | 3-Mercapto-4-methyl-4H- | 2-(2-chlorophenyl)-1-[2-(2- |
| triazole (MTA) | 1,2,4-triazole (MMHTA) | chlorophenyl)-4,5- |
| | | diphenylimidazol-1-yl]-4,5- |
| | | diphenylimidazole (HABI-1) |
| Co-initiator | Co-initiator (triazole) | Photosensitizer |
| (triazole) | | |

Evaluation of HABI-1 with Different Co-Initiators at Constant Mixing Ratios

Camphorquinone was substituted equimolarly by HABI-1. EHA was substituted in the same weight ratio by MTA, MPHTA, HPMTA, and EPMTA (see Table 2).

The PI system HABI-1/MTA is characterized by high flexural strength and a high modulus of elasticity with high double bond conversion, but has a low cure depth compared to the reference (see Table 3b). The HABI-1 systems in combination with MPHTA, HPMTA or EPMTA, on the other hand, are characterized by a slightly lower flexural strength and modulus, but have a higher cure depth compared to HABI-1/MTA (see Tables 3a, 4a and 4b).

TABLE 2
Compositions of formulations with a constant
proportion of o-CI-HABI and co-initiator.

| | Substance | | | Proportion |
|---|---|---|---|---|
| Test no. | class | Designation | Chemical name | [wt.-%] |
| Reference | (ii) and (iii) | EBisA | Ethoxylated bisphenol A | 19.29 |
| | Monomers | | dimethacrylate | |
| | | UDMA | Urethane dimethacrylate | 15 |
| | | TEGDMA | Triethylene glycol | 0.2 |
| | | | dimethacrylate | |
| | PI | CQ | Camphorquinone | 0.04 |
| | | EHA | Ethylhexyl dimethylamino | 0.16 |
| | | | benzoate | |
| | Fillers | YbF3 | Ytterbium fluoride | 15 |
| | | Dental glass | Barium aluminum borofluoride | 49 |
| | | | silicate glass | |
| | | Silica | Silicon dioxide | 1 |
| | Additives | Stabilizer | Butylated hydroxytoluene | 0.03 |
| | | UV additive | Oxybenzone | 0.28 |
| 1.1 | (ii) and (iii) | EBisA | Ethoxylated bisphenol A | 19.21 |
| | monomers | | dimethacrylate | |
| | | UDMA | Urethane dimethacrylate | 14.9 |
| | | TEGDMA | Triethylene glycol | 0.2 |
| | | | dimethacrylate | |
| | PI | HABI-1 | 2,2′-Bis(2-chlorophenyl)-4,4′, | 0.25 |
| | | | 5,5′-tetraphenyl-1,2′-biimidazole | |
| | | MTA | 3-Mercapto-1,2,4-triazole | 0.16 |
| | Fillers | YbF3 | Ytterium fluoride | 15 |
| | | Dental glass | Barium aluminum borofluoride | 49 |
| | | | silicate glass | |
| | | Silica | Silicon dioxide | 1 |
| | Additives | Stabilizer | Butylated hydroxytoluene | 0.03 |
| | | UV additive | Oxybenzone | 0.28 |
| 1.2 | (ii) and (iii) | EBisA | Ethoxylated bisphenol A | 19.21 |
| | monomers | | dimethacrylate | |
| | | UDMA | Urethane dimethacrylate | 14.9 |
| | | TEGDMA | Triethylene glycol | 0.2 |
| | | | dimethacrylate | |
| | PI | HABI-1 | 2,2′-Bis(2-chlorophenyl)-4,4′, | 0.25 |
| | | | 5,5′-tetraphenyl-1,2′-biimidazole | |
| | | MPHTA | 5-Mercapto-1-phenyl-1H- | 0.16 |
| | | | tetrazole | |
| | Fillers | YbF3 | Ytterbium fluoride | 15 |
| | | Dental glass | Barium aluminum borofluoride | 49 |
| | | | silicate glass | |
| | | Silica | Silicon dioxide | 1 |
| | Additives | Stabilizer | Butylated hydroxytoluene | 0.03 |
| | | UV additive | Oxybenzone | 0.28 |
| 1.3 | (ii) and (iii) | EBisA | Ethoxylated bisphenol A | 19.21 |
| | monomers | | dimethacrylate | |
| | | UDMA | Urethane dimethacrylate | 14.9 |
| | | TEGDMA | Triethylene glycol | 0.2 |
| | | | dimethacrylate | |
| | PI | HABI-1 | 2,2′-Bis(2-chlorophenyl)-4,4′, | 0.25 |
| | | | 5,5′-tetraphenyl-1,2′-biimidazole | |
| | | MMHTA | 3-Mercapto-4-methyl-4H-1,2,4- | 0.16 |
| | | | triazole | |
| | Fillers | YbF3 | Ytterium fluoride | 15 |
| | | Dental glass | Barium aluminum borofluoride | 49 |
| | | | silicate glass | |
| | | Silica | Silicon dioxide | 1 |
| | Additives | Stabilizer | Butylated hydroxytoluene | 0.03 |
| | | UV additive | Oxybenzone | 0.28 |
| 1.4 | (ii) and (iii) | EBisA | Ethoxylated bisphenol A | 19.21 |
| | monomers | | dimethacrylate | |
| | | UDMA | Urethane dimethacrylate | 14.9 |
| | | TEGDMA | Triethylene glycol | 0.2 |
| | | | dimethacrylate | |
| | PI | HABI-1 | 2,2′-Bis(2-chlorophenyl)-4,4′, | 0.25 |
| | | | 5,5′-tetraphenyl-1,2′-biimidazole | |
| | | EPMTA | 1-(4-Ethoxyphenyl)-5- | 0.16 |
| | | | mercapto-1H-tetrazole | |
| | Fillers | YbF3 | Ytterium fluoride | 15 |
| | | Dental glass | Barium aluminum borofluoride | 49 |
| | | | silicate glass | |
| | | Silica | Silicon dioxide | 1 |
| | Additives | Stabilizer | Butylated hydroxytoluene | 0.03 |
| | | UV additive | Oxybenzone | 0.28 |
| 1.5 | (ii) and (iii) | EBisA | Ethoxylated bisphenol A | 19.21 |
| | monomers | | dimethacrylate | |
| | | UDMA | urethane dimethacrylate | 14.9 |
| | | TEGDMA | Triethylene glycol | 0.2 |
| | | | dimethacrylate | |
| | PI | HABI-1 | 2,2′-Bis(2-chlorophenyl)-4,4′, | 0.25 |
| | | | 5,5′-tetraphenyl-1,2′-biimidazole | |
| | | HPMTA | 1-(4-hydroxyphenyl)-5- | 0.16 |
| | | | mercapto-1H-tetrazole | |
| | Fillers | YbF3 | Ytterbium fluoride | 15 |
| | | Dental glass | Barium aluminum borofluoride | 49 |
| | | | silicate glass | |
| | | Silica | Silicon dioxide | 1 |
| | Additives | Stabilizer | Butylated hydroxytoluene | 0.03 |
| | | UV additive | Oxybenzone | 0.28 |

TABLE 3a
Results 0.25 wt.-% HABI-1 with 0.16 wt.-% MTA, 0.16
wt.-% MPHTA, and 0.16 wt.-% MMHTA in each case.

| | Curing |
|---|---|
| Test no. | depth [mm] |
| Reference (CQ/EHA) | 13.02 |
| 1.1 MTA | 8.23 |
| 1.2 MPHTA | 10.71 |
| 1.3 MMHTA | 5.61 |
| 1.4 EPMTA | 8.9 |
| 1.5 HPMTA | 9.1 |

TABLE 3b
Additional material properties of the compositions

| | Flexural | Modulus of | Double bond | Curing |
|---|---|---|---|---|
| | strength | elasticity | conversion | depth |
| Test no. | [MPa] | [MPa] | [%] | [mm] |
| Reference | 110 | 4656 | 58.66 | 13.02 |
| (CQ/EHA) | | | | |
| 1 | 152 | 7198 | 67.79 | 8.23 |
| 1.2 | 120 | 6424 | 68.7 | 10.71 |
| 1.3 | 128 | 6591 | 65.8 | 5.61 |
| 1.4 | 127 | 5803 | 63.2 | 8.9 |
| 1.5 | 119 | 6414 | 62.21 | 9.1 |

It can be seen that the cure depth for the combination of HABI and tetrazole is higher than for the combination of HABI and triazole. Compared to the reference (CQ/EHA), significantly better mechanical properties can be obtained for the dental composite according to the invention with the photoinitiator according to the invention.

The co-initiator MTA in combination with HABI-1 (0.2 wt.-%) only results in high mechanical strength of the cured composite material. Only in combination with the mercaptotetrazoles MPHTA, EPMTA, and HPMTA is a significantly higher curing depth achieved compared to MTA. Tables 4a and 4b show the mixing ratios between MTA and MPHTA, EPMTA, and HPMTA.

TABLE 4a
Mixing ratios of MTA/MPHTA, EPMTA, HPMTA with HABI-1.

| No | Component | Reference | 2 | 3 | 4 | 5 | 6 | 7 | 8 |
|---|---|---|---|---|---|---|---|---|---|
| Proportion | EBisA | 19.29 | 19.36 | 19.37 | 19.53 | 19.53 | 19.37 | 19.53 | 19.53 |
| [% by | UDMA | 15.00 | 14.98 | 14.98 | 15.11 | 15.11 | 14.98 | 15.11 | 15.11 |
| weight] | TEGDMA | 0.2 | 0.20 | 0.20 | 0.20 | 0.20 | 0.20 | 0.20 | 0.20 |
| | YbF₃ | 15.00 | 14.95 | 14.96 | 15.00 | 15.00 | 14.96 | 15.00 | 15.00 |
| | Dental glass | 49.00 | 48.35 | 48.37 | 48.5 | 48.5 | 48.37 | 48.5 | 48.5 |
| | Silica | 1.00 | 1.00 | 1.00 | 1.01 | 1.01 | 1.00 | 1.01 | 1.01 |
| | Stabilizer | 0.03 | 0.03 | 0.03 | 0.04 | 0.04 | 0.03 | 0.04 | 0.04 |
| | UV additive | 0.28 | 0.76 | 0.76 | 0.25 | 0.25 | 0.76 | 0.25 | 0.25 |
| | MTA | — | 0.16 | 0.10 | 0.1 | 0.10 | 0.06 | 0.06 | 0.06 |
| | HABI-1 | — | 0.20 | 0.18 | 0.20 | 0.20 | 0.18 | 0.20 | 0.20 |
| | MPHTA | — | | 0.06 | — | — | 0.10 | — | — |
| | EPMTA | — | | — | 0.06 | — | — | 0.1 | — |
| | HPMTA | — | | — | — | 0.06 | — | — | 0.1 |
| | CQ | 0.04 | | — | — | — | — | — | — |
| | EHA | 0.16 | | — | — | — | — | — | — |
| | Triazole/ | — | | 5/3 | 5/3 | 5/3 | 3/5 | 3/5 | 3/5 |
| | Tetrazole | | | | | | | | |
| | [wt.-%/wt.-%] | | | | | | | | |
| | Flexural | 110 | 148 | 131 | 150 | 149 | 137 | 132 | 116 |
| | strength | | | | | | | | |
| | [MPa] | | | | | | | | |
| | Modulus of | 4656 | 6103 | 6647 | 6890 | 6713 | 6563 | 6222 | 6661 |
| | elasticity | | | | | | | | |
| | [MPa] | | | | | | | | |
| | AHT [mm] | 13.02 | 9.10 | 9.03 | 9.0 | 9.4 | 10.08 | 9.7 | 9.7 |

TABLE 4b
Mixing ratios of MTA/MPHTA, EPMTA, HPMTA with HABI-1.

| No | Component | Reference | 2 | 9 | 10 | 11 | 12 | 13 | 14 | 15 |
|---|---|---|---|---|---|---|---|---|---|---|
| Proportion | EBisA | 19.29 | 19.36 | 19.37 | 19.35 | 19.36 | 19.53 | 19.53 | 19.53 | 19.53 |
| [% by | UDMA | 15.00 | 14.98 | 14.98 | 14.97 | 14.98 | 15.11 | 15.11 | 15.11 | 15.11 |
| weight] | TEGDMA | 0.2 | 0.20 | 0.20 | 0.20 | 0.20 | 0.20 | 0.20 | 0.20 | 0.20 |
| | YbF₃ | 15.00 | 14.95 | 14.96 | 14.94 | 14.95 | 15.00 | 15.00 | 15.00 | 15.00 |
| | Dental glass | 49.00 | 48.35 | 48.37 | 48.33 | 48.35 | 48.5 | 48.5 | 48.5 | 48.5 |
| | Silica | 1.0 | 1.00 | 1.00 | 1.00 | 1.00 | 1.01 | 1.01 | 1.01 | 1.01 |
| | Stabilizer | 0.03 | 0.03 | 0.03 | 0.03 | 0.03 | 0.04 | 0.04 | 0.04 | 0.04 |
| | UV additive | 0.28 | 0.76 | 0.76 | 0.76 | 0.76 | 0.25 | 0.25 | 0.25 | 0.25 |
| | MTA | — | 0.16 | 0.04 | 0.04 | 0.04 | 0.08 | 0.04 | 0.08 | 0.04 |
| | HABI-1 | — | 0.20 | 0.18 | 0.25 | 0.20 | 0.20 | 0.20 | 0.20 | 0.20 |
| | MPHTA | — | | 0.12 | 0.12 | 0.12 | — | — | — | — |
| | EPMTA | — | | — | — | — | 0.08 | 0.12 | — | — |
| | HPMTA | — | | — | — | — | — | — | 0.08 | 0.12 |
| | CQ | 0.04 | | — | — | — | — | — | — | — |
| | EHA | 0.16 | | — | — | — | — | — | — | — |
| | Triazole/ | — | | 1/3 | 1/3 | 1/3 | 1/1 | 1/3 | 1/1 | 1/3 |
| | Tetrazole | | | | | | | | | |
| | [wt./wt.] | | | | | | | | | |
| | Flexural | 110 | 148 | 136 | 134 | 126 | 138 | 141 | 144 | 131 |
| | strength | | | | | | | | | |
| | [MPa] | | | | | | | | | |
| | Modulus of | 4656 | 6103 | 6867 | 7011 | 6379 | 6433 | 6924 | 6480 | 6260 |
| | elasticity | | | | | | | | | |
| | [MPa] | | | | | | | | | |
| | AHT [mm] | 13.02 | 9.10 | 10.17 | 10.24 | 9.80 | 9.0 | 10.0 | 9.8 | 9.8 |

At a concentration of greater than or equal to 0.1 wt.-% of mercaptotriazole in combination with HABI, curing depths of greater than or equal to 10 mm can be achieved, particularly in the dental composite with fillers according to the invention.

The data in Tables 3 to 4b show that an optimal mixing ratio [wt.-%/wt.-%] of mercaptotriazole/mercaptotetrazole is 1 to 3 or 3 to 5 with a weight proportion of HABI-1 of approximately 0.2 wt.-%. In this way, a mechanical strength comparable to the reference in terms of flexural strength and modulus of elasticity (up to 149 MPa flexural strength and up to 6900 MPa modulus of elasticity) as well as an acceptable cure depth (up to 10.2 mm) can be achieved in a standard dental composite. Based on the double bond conversion rate, which is comparable to the reference after a standard exposure time of 20 seconds, the photoinitiator system can also be determined to have good polymerization quality. A high double bond conversion rate leads to a high crosslink density of the material and reduces the proportion of soluble components in the medical device.

1. Polymerizable dental composite material curable with light rays, comprising
(i) 30 to 90 wt.-% of an inorganic filler component comprising at least one dental glass and, optionally, at least one amorphous metal oxide,
(ii) 5 to 60 wt.-% comprising at least one urethane acrylate, urethane methacrylate mixtures thereof, or
(iii) 0.01 to 30 wt.-% of at least one di-, tri-, tetra- or multi-functional monomer that is not a urethane acrylate and/or a urethane alkyl acrylate,
(iv) 0.01 to 10 wt.-% comprising hexaarylbisimidazole and/or substituted hexaarylbisimidazole and comprising at least one mercaptotetrazole as an initiator system, the total composition of the composite material being 100 wt.-%.

2. Dental composite material according to claim 1, wherein it comprises
(v) 0.01 to 10 wt.-% of at least one stabilizer and optionally at least one UV additive, wherein the total composition of the composite material is 100 wt.-%.

3. Dental composite material according to claim 1, wherein
(iv) comprising
a) hexaarylbisimidazole, substituted hexaarylbisimidazole and/or mixtures thereof, and
b) at least one mercaptotetrazole comprising phenyl- and/or substituted phenyl-substituted mercaptotetrazole or mixtures thereof as initiator system.

4. Dental composite material according to claim 1, wherein
(iv) comprising
a) hexaarylbisimidazole comprising 2,2′-bis(phenyl)-4,4′,5,5′-tetraphenyl-1,1′-bi-1H-imidazole, and/or substituted hexaarylbisimidazole and/or mixtures thereof, and
b) at least one mercaptotetrazole, and
c) at least one mercaptotriazole and/or substituted mercaptotriazole
as the initiator system.

5. Dental composite material according to claim 1, wherein
(iv) comprising
a) hexaarylbisimidazole and/or substituted hexaarylbisimidazole and/or mixtures thereof, and
b) at least one mercaptotetrazole selected from 1-phenyl-mercaptotetrazoles and p-substituted 1-phenyl-mercaptotetrazoles, and
c) at least one mercaptotriazole as an initiator system.

6. Dental composite material according to claim 1, wherein the
a) substituted hexaarylbisimidazole comprises 2,2′-bis(2-chlorophenyl)-4,4′,5,5′-tetraphenyl-1,1′-bi-1H-imidazole (o-CI-HABI), 2,2′-bis(2,4-dichlorophenyl)-4,4′,5,5′-tetraphenyl-1,1′-bi-1H-imidazole (2,4-CI-HABI), 2,2′-Bis(3-chlorophenyl)-4,4′,5,5′-tetraphenyl-1,1′-bi-1H-imidazole (3-CI-HABI), 2,2′-Bis(4-chlorophenyl)-4,4′,5,5′-tetraphenyl-1,1′-bi-1H-imidazole (4-CI-HABI), 2,2′-Bis(phenyl)-4,4′-bi (2-chlorophenyl)-5,5′-biphenyl-1,1′-bi-1H-imidazole, 2,2′-Bis(phenyl)-4,4′-biphenyl-5,5′-bi (2-chlorophenyl)-1,1′-bi-1H-imidazole, 2,2′-Bis(phenyl)-4,4′,5,5′-tetra(2-chlorophenyl)-1,1′-bi-1H-imidazole and mixtures comprising at least two of the aforementioned hexaarylbisimidazoles.

7. Dental composite material according to claim 1, wherein
b) the at least one mercaptotetrazole comprises 5-Mercapto-1-phenyl-1H-tetrazole (MPHTA), 1-(4-Hydroxyphenyl)-5-mercapto-1H-tetrazole (HPMTA), 1-(4-ethoxyphenyl)-5-mercapto-1H-tetrazole (EPMATA), 1-(4-carboxyphenyl)-5-mercapto-1H-tetrazole, 4-(5-sulfanyl-1H-1,2,3,4-tetrazol-1yl)benzonitrile (STABN), 1-[4-(5-Mercapto-1H-tetrazol-1-yl)-phenyl]ethanone (MTPE) and/or mixtures comprising at least two of the aforementioned tetrazoles.

8. Dental composite material according to claim 4, wherein the
c) at least comprises one mercaptotriazole 3-Mercapto-1,2,4-triazole (MTA), 3-Mercapto-4-methyl-4H-1,2,4-triazole and/or mixtures thereof.

9. Dental composite material according to claim 4, wherein
(iv) comprising
a) 0.1 to 0.5 wt.-% hexaarylbisimidazole and/or substituted hexaarylbisimidazole and/or mixtures thereof, and
b) 0.02 to 0.2 wt.-% of at least one mercaptotetrazole, and
c) 0.02 to 0.2 wt.-% of at least one mercaptotriazole and/or substituted mercaptotriazole, wherein the total composition of the composite material is 100 wt.-%.

10. Dental composite material according to claim 4, wherein
(iv) comprising
a) 0.15 to 0.3 wt.-% of hexaarylbisimidazole and/or substituted hexaarylbisimidazole and/or mixtures thereof, and
b) 0.04 to 0.2 wt.-% of at least one mercaptotetrazole, and
c) 0.03 to 0.1 wt.-% of at least one mercaptotriazole and/or substituted mercaptotriazole, wherein c) and b) are present in a weight ratio of 5:3 to 3:5, preferably in a weight ratio of 1:3 to 3:5, wherein the total composition of the composite material is 100 wt.-%.

11. Dental composite material according to claim 4, wherein
(iv) comprising
a) 0.15 to 0.3 wt.-% of hexaarylbisimidazole and/or substituted hexaarylbisimidazole and/or mixtures thereof, and
b) 0.06 to 0.2 wt.-% of at least one mercaptotetrazole, and
c) 0.03 to 0.1 wt.-% of at least one mercaptotriazole and/or substituted mercaptotriazole, wherein c) and b) are present in a weight ratio of 5:3 to 3:5, and a:(b+c) are present in a weight ratio of 1:1 to 2:1, wherein the total composition of the composite material being 100 wt.-%.

12. Dental composite material according to claim 1, wherein
(iv) comprising
a) 0.15 to 0.3 wt.-% of hexaarylbisimidazole and/or substituted hexaarylbisimidazole and/or mixtures thereof, and
b) 0.08 to 0.2 wt.-% of at least one mercaptotetrazole, and
c) 0.03 to 0.08 wt.-% of at least one mercaptotriazole and/or substituted mercaptotriazole, wherein c) and b) are present in a weight ratio of 1:3 to 3:5, and a:(b+c) in a weight ratio of greater than or equal to 1.1:1, wherein the total composition of the composite material is 100 wt.-%.

13. Dental composite material according to claim 4, wherein the polymerized composite material has a flexural strength greater than or equal to 130 [MPa] and a modulus of elasticity greater than or equal to 6500 [MPa], determined in accordance with DIN EN ISO 4049:2019.

14. Dental composite material according to claim 1, wherein the curing depth or polymerization depth in mm of the polymerized composite material is greater than or equal to 9.0 mm, wherein the curing depth or polymerization depth is determined according to DIN EN ISO 4049:2019, 7.10, and wherein the curing or polymerization takes place for 20 seconds with a light source, with emission maxima in the wavelength range from 440 to 480 nm and a light output greater than or equal to 1000 mW/cm².

15. Polymerized dental composite material obtainable by polymerization of the composite material according to claim 1, i) with a UV and/or VIS radiation source, preferably with a VIS radiation source with emission maxima in the spectral range from 400 nm to 530 nm.

16. Polymerized dental composite material according to claim 15, comprising
(i) 30 to 90 wt.-% of an inorganic filler component comprising at least one dental glass and, optionally, at least one amorphous metal oxide,
(ii) 5 to 60% by weight comprising at least one polymer based on at least one urethane acrylate, urethane methacrylate or mixtures thereof, and
(iii) 0.01 to 30 wt.-% of at least one di-, tri-, tetra- or multi-functional
monomer which is not a urethane acrylate and/or a urethane alkyl acrylate, and optionally
(iv) 0.01 to 10 wt.-% comprising hexaarylbisimidazole and/or substituted hexaarylbisimidazole and comprising at least one mercaptotetrazole as an initiator system and optionally at least one pigment and optionally at least one stabilizer and optionally at least one UV additive, the total composition of the composite material being 100 wt.-%.

17. Method of using a dental composite material according to claim 1 for producing dental filling materials, dental prosthetic restorations in additive processes and/or material-removing processes, or for producing composite material for the production of direct adhesive dental restorations, hoof repair material, bone cement, bone cement for cementing artificial joint prostheses, orthodontic appliances and/or instruments.

18. (canceled)

19. Dental composite material according to claim 5, wherein
c) at least comprises one mercaptotriazole 3-Mercapto-1,2,4-triazole (MTA), 3-Mercapto-4-methyl-4H-1,2,4-triazole and/or mixtures thereof.

20. Dental composite material according to claim 6, wherein
c) at least comprises one mercaptotriazole 3-Mercapto-1,2,4-triazole (MTA), 3-Mercapto-4-methyl-4H-1,2,4-triazole and/or mixtures thereof.

21. Dental composite material according to claims 4 to 9, characterized in that
(iv) comprising
a) 0.15 to 0.3 wt.-% of hexaarylbisimidazole and/or substituted hexaarylbisimidazole and b) 0.04 to 0.2 wt.-% of at least one mercaptotetrazole, and
c) 0.03 to 0.1 wt.-% of at least one mercaptotriazole and/or substituted mercaptotriazole, wherein c) and b) are present in a weight ratio of 5:3 to 3:5, wherein the total composition
of the composite material is 100 wt.-%.
